(19) United States
(12) Patent Application Publication
(10) Pub. No.: US 2016/0113966 A1
(43) Pub. Date: Apr. 28, 2016

(54) COMPOSITION AND METHODS OF CULTURE SUPERNATANT OF KHYG-1 CELLS

(71) Applicant: Hans Klingemann, Boston, MA (US)

(72) Inventor: Hans Klingemann, Boston, MA (US)

(21) Appl. No.: 14/923,673

(22) Filed: Oct. 27, 2015

Related U.S. Application Data
(60) Provisional application No. 62/122,617, filed on Oct. 27, 2014.

(51) Int. Cl. A61K 35/17; A61K 38/21; A61K 38/20; C12N 5/0783; A61K 9/00

(52) U.S. Cl. CPC A61K 35/17; C12N 5/0646; A61K 9/0019; A61K 38/2013; A61K 38/217; A61K 38/2086; C12N 2501/2302; C12N 2501/2315; C12N 2501/24; C12N 2500/30

(57) ABSTRACT

Provided herein are pharmaceutical compositions comprising tumoricidal and/or antimicrobial components isolated from the supernatant of KHYG-1 cell medium and methods of using the compositions for killing cancer cells and infected cells in a human patient or animal.

### FIELD OF THE INVENTION

This invention relates generally to pharmaceutical compositions having tumoricidal and/or antimicrobial properties and methods of using these compositions for killing cancer, microbes and viruses.

### STATE OF THE ART

Cancer is the leading cause of death in humans as well as in many domesticated animals. Although chemotherapy and radiation and recently immunotherapy have some degree of efficacy in controlling the growth of tumors, those treatments rarely eradicate the cancer completely and regrowth is common with progressive disease, metastases and finally death of the patient. In addition to the lack of complete tumor eradication, these treatments have side effects at times so severe that they affect the quality of life of the person or animal.

Animals are also prone to all types of cancers, especially lymphomas, histiocytoma, melanoma, squamous cell carcinoma, and mast cell tumors.

Despite the advent of new antibiotics, infections continue to be a major problem throughout the world. An increasing number of infections have become resistant to antimicrobials and antiviral drugs. The CDC reports that over two million Americans are infected by antibiotic-resistant microbes every year, resulting in more than 23,000 deaths. Especially in immunocompromised or older patients those drug resistant infections can be life-threatening. Infections are also common in domesticated animals and can be difficult to treat.

Accordingly, there is a clear need for tumoricidal compositions useful in lysing cancer cells, especially those cancer lesions that are accessible to manipulation and direct injection of the compositions. This may include but is not restricted dermal and sub-dermal cancers. There also remains a need for novel antimicrobial compositions useful in treating infection. Similarly such a tumoricidal and antimicrobial composition is useful for treating cancerous lesions and infections in animals as well.

### SUMMARY OF THE INVENTION

Cells of the mammalian immune system such as lymphocytes have cytotoxic activity against a range of target cells including malignant and infected cells. The lymphocyte population contains T-cells and natural killer (NK) cells, generally representing about 10-15% of circulating lymphocytes. Lymphocytes (T and B cells) belong to the adaptive immune system requiring a few days to be primed and be reactive to the target. NK cells on the other hand, belong to the innate immune system and bind and kill target cells, including virus-infected cells and malignant cells, without prior sensitization. Killing of target cells occurs by inducing cell lysis and inhibition of regrowth of target cells. Both T-lymphocytes and NK cells have been shown to be effectively control cancer in patients.

Cytotoxic cells both in humans and animals kill their target by different mechanism: direct contact elicits the release of perforin form intracellular granules that can make the target cell membrane porous so that enzymes such as granzyme can enter the target cells and destroy the DNA. In addition to those enzymes, cytotoxic cells can produce cytokines such as interleukins and interferons that can affect the target cells directly or recruit secondary immune cells and support their activation.

Cells, including cytotoxic blood cells, release a variety of microparticles into the medium in which they are grown. Those microparticles contain molecules such as proteins, RNA cytokines and other small and large molecules. Those molecules are released through expulsion of microvesicles from the cells. Those microvesicles can have different sizes. When they are smaller than 100 nm, they are called exosomes. Those microvesicles are very common in all types of cells and in addition to cytotoxic cells, microvesicles are released by a variety of normal and tumor cells. The products of microvesicles can be detected and isolated from cell culture supernatants and from body fluids (e.g., blood).

Microvesicles isolated from the cell culture supernatant of cytotoxic blood cells contain proteins including perforin, granzyme, FasL, and Rab5B. However, microvesicles and their factors derived from endogenous cytotoxic cells are highly variable, both with regard to the amount of microvesicles recovered and the proteins contained therein. It is believed that this variability arises because endogenous cytotoxic cells represent a heterogeneous population of cells. Differences in conditions such as cell culture conditions, purification methods, initial cell populations, and the ratios of cell types in culture can all lead to variability in the composition and amount of microvesicles that can be purified from endogenous cytotoxic cells.

The KHYG-1 cell line is a unique cell line that was isolated from a patient with a lymphoma. It proliferates without limitations in the presence of interleukin 2 (IL-2). KHYG-1 cell have high cytolytic activity against a variety of cancers. The KHYG-1 cell line is a homogeneous cell population as it relates to its lysing activity. It has characteristic surface antigen expression and target cell killing ability reminiscent of NK cells. However it is different from blood NK cells and other human NK cell “like” cell lines: KHYG-1 cells produces granzyme M to degrade the DNA of the target cells which is not produced by blood NK cells or NK like cell lines cells. KHYG-1 also produces a different spectrum of cytokines.

Endogenous cytotoxic cells are significantly different from KGYG-1 cells, in large part because of their distinct origins: KGYG-1 is a cancer-derived cell line, whereas endogenous cytotoxic lymphocyte cells are harvested from a donor (or the patient) and processed for infusion into a patient. This means that the type of microparticles they produce into any culture supernatant is different from that of a continuously growing and reproducing cell line.

Because KHYG-1 is a cell line, large numbers of cells can be cultured and propagated for extended periods of time. These cultures are homogeneous cell populations that provide consistent, reproducible macrovesicle preparations.

On the other hand, KHYG-1 cells are a cancer cell line. Many cancer cells release microparticles and other factors that are distinct from those released by their non-malignant counterparts. In one embodiment, the microparticles obtained from KHYG-1 cells are incubated in a suitable solution, such as PBS or isotonic saline, to extract such factors prior to their use. Alternatively, only the molecules with cytolytic and antimicrobial activity are separated from the microparticles.

One aspect of the invention provides pharmaceutical compositions comprising one or more components obtained from the supernatant of KHYG-1 cell medium. The components may be tumoricidal and/or anti-microbial. Components may also have immunomodulatory properties. These components are preferably microvesicles and the smaller exosomes isolated from the supernatant. In some embodiments, the pharmaceutical compositions may further comprise one or more pharmaceutically acceptable excipient. In one embodiment, the microvesicles have the ability to lyse cancer cells. In one embodiment, the microvesicles are antimicrobial. In one embodiment, the microvesicles are immunomodulatory. In a preferred embodiment, the pharmaceutical composition does not comprise living cells. In some embodiments, the pharmaceutical composition is an injectable composition. In some embodiments, it is contemplated that the injection will provide systemic immunomodulatory activity.

These and other aspects of the invention will be set forth in details below.

### BRIEF DESCRIPTION OF THE DRAWINGS

FIG. 1 shows the effect of the enriched culture supernatant of MCF-7 breast cancer cells (control) and KHYG-1 cells on the killing of K562 target cells as measured by the number of dead cells (“PI positive”). The blue bar represents data after 2 hour of exposure and the red bar represents killing of K562 after 20 hours of exposure. The MCF cells were cultured in serum free medium (RPMI). KHYG-1 were cultured either in RPMI or X-vivo 10 medium. Both are serum free and hence do not contain microparticles.

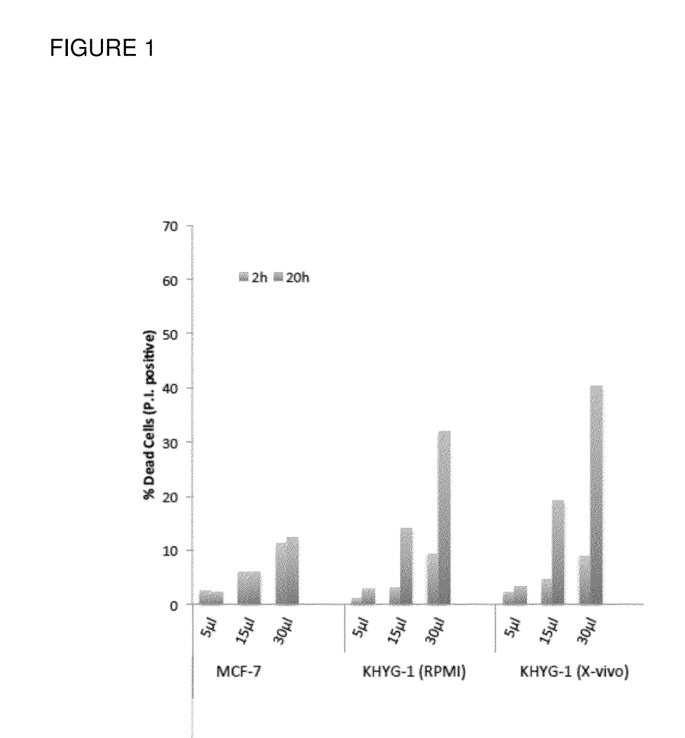

There is significantly higher cytotoxic activity by the KHYG-1 supernatant against the K562 cells than by MCF-7 supernatant which serves as a control.

FIG. 2 shows the similar culture conditions as in FIG. 1, except that apoptosis of K562 and Jurkat cells was measured using an annexing assay. An additional control in the form of PBS was added and for KHYG-1 cells, culture supernatant in the presence of RPMI was tested only.

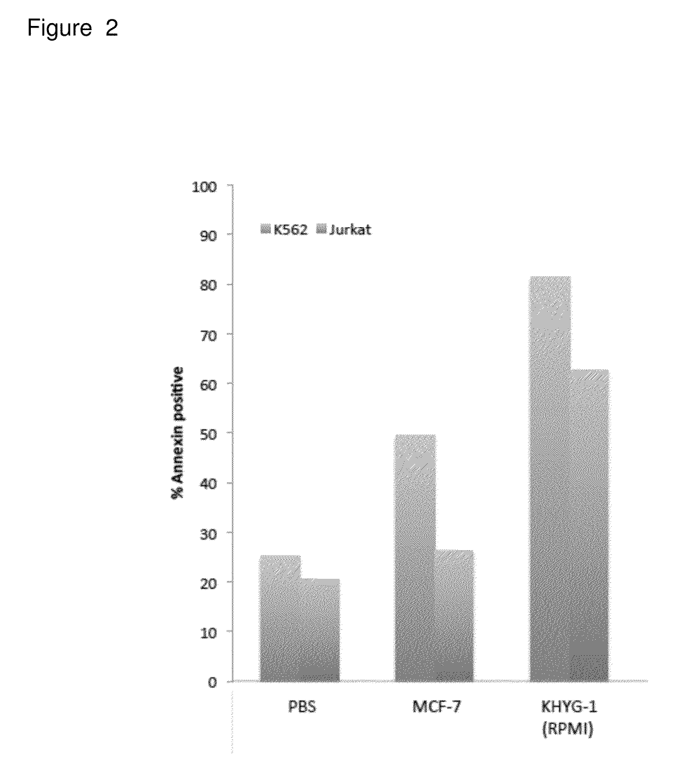

There is significantly higher apoptosis of the K562 and Jurkat cells by KHYG-1 supernatant that by the control PBS.

### DETAILED DESCRIPTION OF THE INVENTION

### Definitions

Before the present articles and methods are disclosed and described, it is to be understood that the aspects described below are not limited to specific compositions, preparation methods, or uses as such may vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a cross-linking agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally cleaning step” means that the cleaning step may or may not be performed.

The term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “patient” as used herein is any vertebrate organism including but not limited to mammalian patients such as humans, farm animals, domesticated pets and the like.

The term “about” when used before a numerical value indicates that the value may vary within a reasonable range, such as ±5%, ±1%, and ±0.2%.

The term “KHYG-1 cell” includes both natural KHYG-1 cells and modified KHYG-1 cells.

As used to describe the present invention, “cancer”, “tumor”, and “malignancy” all relate equivalently to a hyperplasia of a tissue or organ. If the tissue is a part of the lymphatic or immune system, malignant cells may include non-solid tumors of circulating cells. Malignancies of other tissues or organs may produce solid tumors. In general, the methods of the present invention may be used in the treatment of lymphatic cells, circulating immune cells, and solid tumors.

The term “tumoricidal component” refers to components that treat cancerous tumors and/or cancer cells. Treatment of tumors encompasses reducing or eliminating the tumor, killing cancer cells, and/or inhibiting the growth, proliferation, and/or metastasis of cancer cells. Preferably, the tumor cells are killed, for example by cytolysis.

The term “anti-microbial component” refers to components that treat or prevent infection by microbes. Microbes include bacteria, fungi, molds, viruses, etc.

As used to describe the present invention, the terms “cytotoxic” and “cytolytic”, when used to describe the activity of effector cells such as KHYG-1 cells, are intended to be synonymous.

The term “growth medium” or “medium” as used herein are intended to be synonymous. In general, the terms refer to any medium or aqueous solution in which the KHYG-1 cells may be grown or placed and into which the KHYG-1 cells release microvesicles and exosomes or other active components. Medium may comprise growth mediums (e.g., commercially available medium) such as X-Vivo or RPMI. Alternatively, medium may comprise PBS or other aqueous solution.

Components Isolated from the Supernatant of a Medium of KHYG-1 Cells

KHYG-1 cells can be expanded, modified and/or maintained in culture medium. Any acceptable culture conditions may be used. In one embodiment, KHYG-1 cells are cultured in enriched alpha minimum essential medium (MEM; Sigma Chemical Co., St. Louis, Mo.) supplemented with fetal calf serum (for example, at 12.5%; Sigma Chemical Co., St. Louis, Mo.), and/or horse serum (for example, at 12.5%; Sigma Chemical Co., St. Louis, Mo.). In another embodiment, the KHYG-1 cells are cultured in X-Vivo 10 medium supplemented with human serum, human plasma, or human serum albumin (for example, at 5%). The medium optionally is supplemented with other nutrients, cytokines, and/or growth factors, for example interleukin 2 (IL-2), L-asparagine, L-glutamine, and/or L-serine.

To determine the quantity and activity of microvesicles in the culture supernatant, the KHYG-1 cells have to be transferred into culture medium which has to be serum free as serum can contain variable amounts of microvesicles and exosomes. This culture period under serum free conditions is at least 12 hours. Before being placed in serum free medium, the cells are centrifuged and the pellet is washed repeatedly in PBS. Alternatively serum containing medium can be used but it has to be ultracentrifuged to remove any microvesicles and exosomes.

When placed in the serum free medium, KHYG-1 can release components such as proteins, cytokines (such as, for example, IL-2), microvesicles and exosomes into the medium. After the cells are isolated from the medium, for example, by centrifugation, the components released from the cells remain in the supernatant.

In some embodiments, one or more stimulatory agents are added to the medium. It is believed that stimulation of KHYG-1 cells with such agents may result in more consistent, robust, and/or reproducible release of components, including microvesicles and exosomes. Stimulatory agents include, for example, cytokines or pharmaceutical stimulators. In one embodiment, the stimulatory agent is IL-15. In one embodiment, the stimulatory agent is interferon gamma.

In some embodiments, the KHYG-1 cells comprise KHYG-1 cells modified to express a cytokine. In some embodiments, the KHYG-1 cells comprise KHYG-1 cells modified to express a cytokine that promotes growth of the cells and/or a cytokine receptor. In some embodiments, the KHYG-1 cells comprise KHYG-1 cells modified to express IL-2 and/or IL-2 receptor. In some embodiments, the KHYG-1 cells comprise KHYG-1 cells modified to express IL-15, IL-18, or IL-21, or a receptor thereof. Further still, other modified KHYG-1 cells may express an Fc-receptor, a cytokine, a cytokine receptor and/or a chimeric antigen receptor.

In some embodiments the KHYG-1 express a Fc receptor (CD16) or any other receptor on the surface that is not present on the original cell line and has been introduced to the surface either by genetic transduction or chemical/physical step.

### Pharmaceutical Compositions

In one aspect, provided is a pharmaceutical composition useful for killing cancer cells in a warm-blooded animal, which composition comprises a pharmaceutically acceptable carrier and one or more components isolated from the supernatant of a growth medium of KHYG-1 cells.

In some embodiments, the pharmaceutical composition is liquid at room temperature and a gel when applied to the patient. In some embodiments, the pharmaceutically acceptable carrier comprises a poloxamer.

In another aspect, provided is a pharmaceutical composition comprising microvesicles and exosomes isolated from the supernatant (by for example, centrifugation or ultra-centrifugation to cause the microvesicles and exosomes to form a pellet and come out of the suspension of the supernatant) of a growth medium of KHYG-1 cells and a sterile aqueous carrier.

In another aspect, provided is a kit comprising a first pharmaceutical composition comprising a pharmaceutically acceptable carrier and exosomes isolated from the supernatant of a growth medium of KHYG-1 cells, and a second pharmaceutical composition comprising a gel, wherein the first pharmaceutical composition and the second pharmaceutical composition are topical formulations (including, but not limited to a cream or lotion). In some embodiments, the second pharmaceutical composition is a poloxamer. In some embodiments, the second pharmaceutical composition comprises microvesicles and exosomes isolated from the supernatant of a growth medium of KHYG-1 cells.

In some embodiments, the first pharmaceutical composition comprises a liquid and the second pharmaceutical composition comprises a gel, poloxamer, or composition that is a liquid at room temperature and a gel at body temperature. The liquid is applied to the treatment area first, and the gel is applied over the liquid. It is believed that the liquid formulation provides rapid treatment of the area, while the gel maintains the liquid at the site of application. In some embodiments, the gel comprises microvesicles and exosomes isolated from the supernatant of a growth medium of KHYG-1 cells and a sterile aqueous carrier. It is believed that the gel will provide slower release of this micro-particles and its components to the treatment area, thus providing for sustained release and treatment of the affected area.

In some embodiments, the pharmaceutical composition is an injectable extended release formulation. The tumoricidal and/or antimicrobial components in the extended release formulation can be released from the composition to the body over an extended period of time, such as over at least several minutes, at least one hour, at least several hours, at least one day, at least several days, or in weeks, etc. to provide long term and/or continuous therapeutic effects

The pharmaceutical composition can be in a variety of formulations suitable for oral, topical, transdermal, rectal, inhalation, or parenteral (intravenous, intramuscular, or intraperitoneal) administration, and the like.

As used herein “pharmaceutically acceptable carrier” or “pharmaceutically acceptable excipient” includes any and all solvents, dispersive agents or media, coating(s), antimicrobial agents, iso/hypo/hypertonic agents, absorption-modifying agents, and the like, suitable for pharmaceutical use and compatible with the tumoricidal and/or antimicrobial components. Moreover, other or supplementary active ingredients can also be incorporated into the final composition.

The pharmaceutical compositions described herein can be administered in a number of ways depending on whether local or systemic treatment is desired, and on the area to be treated. In one aspect, administration can be by injection, or where the composition is formulated into a liquid or gel and can be applied topically (including ophthalmically, vaginally, rectally, intranasally, orally, or directly to the skin).

In some embodiments, the pharmaceutical composition is an injectable formulation.

The pharmaceutical composition may be administered parenterally, e.g., intravenously, intramuscularly, intravenously, subcutaneously, or interperitonically. The composition can be injected systematically or locally to or near the site of a cancer. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable compositions are prepared by incorporating the tumoricidal and/or antimicrobial components in a suitable amount into an appropriate carrier.

### Method of Treatment of the Invention

The compositions described herein are useful in treating a variety of diseases, such as cancer. Examples of diseases that can be treated by the compositions include malignancies of the immune system, the lymphatic system, and the hematopoietic system, formed tumors, and solid tumors. Non-limiting examples of cancers that can be treated with the compositions include mast cell leukemia, acute myelogenous leukemia (AML), erythroleukemia, myeloid disorders (e.g., myeloid leukemia, multiple myeloma, and erythroleukemia), germ cell tumors, lung carcinoma, small-cell lung carcinoma, gastrointestinal stromal tumors, neuroblastoma, cervical carcinoma, ovarian carcinoma, brain carcinoma, breast carcinoma, ovary carcinoma, endometrium carcinoma, kidney carcinoma, thyroid carcinoma, bladder carcinoma, colon carcinoma, pancreas carcinoma and prostate carcinoma, skin carcinoma such as melanoma, adenomas (e.g., villous colon adenoma), and sarcomas (e.g., osteosarcoma), etc.

In some embodiments, the pharmaceutical compositions described herein are used to treat skin cancer, for example melanoma, squamous cell cancer, basal cell cancer, or mast cell tumors.

In some embodiments, the pharmaceutical compositions described herein are used to treat other epithelial cell cancers, for example cervical cancer or oral cancer. In some embodiments, the pharmaceutical compositions described herein are used to treat virus-infected cells, for example warts.

In some embodiments, the pharmaceutical compositions described herein are used to modulate the immune system. For example, microvesicles and exosomes may induce cytotoxic T cell response (e.g., against tumors), and/or induce apoptosis (e.g., of activated immune cells). Microvesicles and exosomes may also play a role in immune surveillance.

In some embodiments, the pharmaceutical compositions described herein are useful in treating infections. In one embodiment, the pharmaceutical compositions described herein are useful in treating infections by pathogenic viruses, such as HPV, HIV, EBV, CMV, and herpes. In one embodiment, the pharmaceutical compositions described herein are useful in treating bacterial infections. In one embodiment, the pharmaceutical compositions described herein are useful in treating infections caused by other microbes, including fungus.

With mammals, including humans and domesticated animals, the effective amounts can be administered on the basis of body surface area. A suitable dose range is from 0.001 mg to 100 mg of equivalent per m² body surface area of a tumoricidal and/or antimicrobial component, for instance from 0.005 to 50 mg/m². The dosage can be administered daily, such as once, twice, three times or more per day, or every two or several days, or every week, etc. The frequency of administration can be reduced if an extended release formulation is administered.

The dosage and frequency of administration may depend on the type of formulation, the disease being treated, the amount of the tumoricidal and/or antimicrobial component, the patient's age, gender, species, other conditions, etc.

### Combination Therapy

In another aspect, the compositions described herein can be administered in conjunction with other cancer therapies, such as surgery, radiation, chemotherapy (e.g., cisplatin, carboplatin, oxaliplatin, satraplatin, and picoplatin, especially cisplatin and carboplatin; taxanes, such as paclitaxel and docetaxel; and anthracyclines such as daunorubicin, doxorubicin, epirubicin, idarubicin, or valrubicin, etc.), cell based therapy (e.g., KHYG-1 cell therapy), etc. In some embodiments, the composition can be administered in conjunction with one or more cytokines as described herein.

The following examples are included to illustrate the invention and not to limit the invention. All publications or references cited in the present specification are hereby incorporated by reference.

What is claimed is:

1. A method of preparing a composition of isolated microvesicles and molecules from a KHYG-1 cell culture comprising the steps of:
obtaining a quantity of KHYG-1 cells;
culturing the quantity of KHYG-1 cells in a serum free medium, the culturing being done for a time period greater than 12 hours;
centrifuging the KHYG-1 cell culture, the centrifuging removing the quantity of KHYG-1 from a supernatant culture medium formed during the culturing step.

2. The method of claim 1 further comprising the step of adding a stimulatory agent during the culturing step, the stimulatory agent being at least one of IL-2, IL-15, and interferon gamma.

3. The method of claim 1 wherein the step of obtaining the quantity of KHYG-1 comprises obtaining KHYG-1 cells modified to express at least one of a Fc-receptor, a cytokine, a cytokine receptor, and a chimeric antigen receptor.

4. The method of claim 1 further comprising the step of adding a gelling agent to the supernatant culture medium configured to make the supernatant culture medium in a gel form at a body temperature.

5. The method of claim 4 wherein the gelling agent is a poloxamer.

6. The method of claim 1 further comprising the steps of separating the supernatant culture medium into two portions, and adding a gelling agent to only a first portion of the supernatant culture medium configured to make the supernatant culture medium in a gel form at a warm-blooded animal body temperature.

7. The method of claim 1 further comprising the step of ultra-centrifuging the supernatant culture medium to obtain a pellet of microvesicles from the supernatant culture medium.

8. The method of claim 1 further comprising the step of adding one or more pharmaceutically acceptable excipients to the supernatant culture medium.

9. The method of claim 8 further comprising the step of administering the supernatant culture medium to a warm-blooded patient.

10. The method of claim 9 wherein the step of administering comprises injecting the supernatant culture medium.

11. The method of claim 6 further comprising a step of administering the first portion of the supernatant culture medium, and a second of the two portions of the supernatant culture medium to a same area of a warm-blooded patient.

12. The method of claim 11 wherein the area of administration is a cancerous tumor.

13. The method of claim 7 further comprising the step of suspending the microvesicles of the pellet in a second serum free medium.

14. A pharmaceutical composition comprising:
a plurality of isolated microvesicles and molecules produced from KHYG-1 cells;
a sterile, serum-free aqueous solution, the plurality of isolated microvesicles and molecules suspended in the sterile, serum-free aqueous solution; and
at least one pharmaceutically acceptable excipient.

15. The pharmaceutical composition of claim 14 further comprising a gelling agent configured to cause the pharmaceutical composition to be in a gel form at a warm-blooded animal body temperature.

16. The pharmaceutical composition of claim 15 wherein the gelling agent is a poloxamer.

17. The pharmaceutical composition of claim 14 wherein the sterile, serum-free aqueous solution is a supernatant of a KHYG-1 cell culture.

18. The pharmaceutical composition of claim 14 wherein the plurality of isolated microvesicles and molecules comprises at least IL-2.

19. The pharmaceutical composition of claim 14 wherein the plurality of isolated microvesicles and molecules comprises one or more cytokines.

20. The pharmaceutical composition of claim 14 wherein the sterile, serum-free aqueous solution further comprises at least one of IL-2, IL-15, and interferon gamma.
